(19) United States
(12) Patent Application Publication
(10) Pub. No.: US 2026/0130663 A1
(43) Pub. Date: May 14, 2026

(54) SOFT ANCHOR SYSTEMS

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Belin Mirabile, Boston, MA (US); Benjamin M. Hall, Roslindale, MA (US); Mark E. Housman, North Attleboro, MA (US); Christopher D. MacCready, Medfield, MA (US); Jay A. Shah, Brighton, MA (US); Marc J. Balboa, Hopkinton, MA (US); Jon-Paul Rogers, North Smithfield, RI (US)

(21) Appl. No.: 19/443,160

(22) Filed: Jan. 8, 2026

Related U.S. Application Data
(63) Continuation of application No. PCT/US24/39589, filed on Jul. 25, 2024.
(60) Provisional application No. 63/529,041, filed on Jul. 26, 2023.
(60) Provisional application No. 63/604,942, filed on Dec. 1, 2023.

(51) Int. Cl. A61B 17/04

(52) U.S. Cl. CPC A61B 17/0401; A61B 17/0485; A61B 17/0487; A61B 2017/0464

(57) ABSTRACT

A knotless tissue repair construct. The construct includes an anchor body formed of a soft suture-like material. The construct includes a repair suture having a first end, a second end and a cannulated length portion therebetween, the first end fixedly coupled to the anchor body. The cannulated length is interwoven along and through a first sidewall of the anchor body such that the second end extends proximally from the anchor body proximal end. The construct includes a transfer suture threaded through and along a second sidewall of the anchor body, through and along the repair suture cannulated length and also punched through the repair suture adjacent the cannulated length, defining a repair suture punch.

### CROSS-REFERENCE TO RELATED APPLICATIONS

This application is a US continuation application of U.S. PCT application serial number PCT/US24/39589 filed Jul. 25, 2024, titled “SOFT ANCHOR SYSTEMS’ which claims priority to which claims the benefit of U.S. provisional application Ser. No. 63/529,041 filed Jul. 26, 2023, and titled, “SUTURE STOPPER KNOT CONSTRUCT”; and U.S. provisional application Ser. No. 63/604,942 filed Dec. 1, 2023, and titled, “SOFT ANCHOR KNOTLESS SYSTEM.” These provisional applications are all incorporated by reference herein, in their entirety as if reproduced in full below.

### FIELD

The present disclosure relates to systems and methods associated with a repair system that may include a knotless soft anchor system.

### BACKGROUND

Many orthopedic surgeries involve the use of anchoring devices in procedures for attaching tissues, such as soft tissue to bone. Such procedures include, for example, attaching tendons to bone, bone to bone, tendon to tendon, ligaments or grafts to bone, as well as augmenting a primary repair and ligament reconstruction. Generally, these procedures rely on the use of polymeric, metal, or biodegradable rigid anchors with suture attached. The suture is passed through the tissue and a knot secures the anchor and tissue together. However, the use of these anchors often requires rigid, hard materials to be placed in tissue such as bone. If the anchors loosen, a surgeon or surgical technician is faced with the problem of having a potentially hard device migrate into a patient's joint, placing the patient at risk for arthritis. Accordingly, an anchor formed from only soft materials may pose less risk of anatomical damage in a joint or body cavity, should they be dislodged post-operatively.

Existing soft material anchors available today may be formed of suture or soft flexible material, that may include a braided body. These soft anchors may anchor with tissue by deforming or, in some cases, simply relaxing to a radially or laterally expanded state which may be locked in this anchored state by tying knots. Knot-tying may however add complexity to the procedure, may require a higher level of expertise, may be time consuming, and/or be more anatomically disruptive to surrounding structures. Adding knots may also tend to form what is called a knot stack, which may add bulk and palpability at the repair site. Therefore, there is a need for a soft anchor system that includes a knotless locking construct, avoiding the step of tying a knot and addressing the issues listed herein.

During deployment of these soft material anchors, they may deform to embed into a target tissue. Higher loads to deploy the anchor, oftentimes via higher loads on the deploying suture(s), may provide stronger anchor fixation. However, these higher deployment loads may also adjust locations and lengths of portions of the anchoring construct, which may inadvertently affect the final knotless fixation strength of the anchor system.

For example, consider a previously disclosed example knotless construct, illustrated in FIG. 1A-1B. FIG. 1A depicts the example knotless construct 100 as obtained. In this configuration, the soft anchor 102 is an tubular body in an elongate configuration. The transfer suture 120 may be routed through a cannulation 137 of the repair suture 130, to define and form the suture locking passage portion 147 of the construct 100, during later operation of said construct 100. Locking passage portion 147 may extend along the anchor 102, and also interweave repeatedly between the braided strands of the anchor wall. Locking passage portion 147 is shown extending from entrance 137a to exit 137b of the repair suture cannulation 137. In use, repair suture end 136 may be coupled to a target repair tissue and then coupled to transfer suture end 122a. The transfer suture 120 may then shuttle repair suture end 136 back through the anchor 102 and through locking passage 147 to form a knotless locking loop construct. Details on this construct and operation thereof as well as other example prior art constructs are disclosed in commonly owned International patent application number PCT/US2022/048644, filed Nov. 2, 2022, and titled “KNOTLESS SOFT ANCHOR SYSTEM”, herein incorporated by reference in its entirety.

Repair suture 130 may fixedly couple at a distal end of anchor 102 via a double half hitch knot 132 that axially overlaps. Knot 132 is large enough to prevent the repair suture 130 from being pulled between the braided wall of the anchor 102 and therefore prevents the suture 130 from being detaching from the anchor 102. Knot 132 may be disposed distal to the anchor distal end 112.

Turning now to FIG. 1B, showing a close up of the knot 132 with the anchor removed from the figure for simplicity of understanding. The inventors have found that tension on repair end 136 may increase the length “L”, defined as between the top end location of the knot 132 and passage entrance 137a. Tension may be applied to end 136 on three different occasions, that may all increase this length “L”; firstly, during anchor deployment, secondly while knotlessly locking the repair loop (cinching the finger trap) and thirdly during cyclic use as the patient moves after the procedure. The increase in length “L” may be due to knot slippage and/or knot tightening during deployment, thereby moving the knot location relative to entrance 137a. In addition, the suture length “L” may elongate due to these loads. Subsequent cyclic loads can occur post-op, due to loading of the repair construct during use throughout the healing period, further tightening the knot and/or elongating the suture length “L”. This increase in length “L” may reduce the knotless locking strength of the locking passage portion 147, as this finger trap mechanism relies on tension to maintain the outer tubular wall of repair suture 130 along the locking passage 147 in an elongate and reduced diameter configuration. In addition, this increase in length “L” may allow the proximal end of locking passage (137b) to move proximally and out of the anchor 102, causing some clinician concern and/or possibly irritation of the local tissues. Disclosed herein are various methods and suture constructs to limit an increase in length “L”.

In some repairs, there is a need for a low-profile soft anchor system that may couple to multiple locations, in some cases without the need to tie a knot. The multiple locations may be along a tissue or tissues, each location coupled independently. Unlike rigid anchor systems, that tend to retain their outer profile with the addition of sutures, adding sutures and/or knotless construct to soft anchors generally adds volume and bulk to the soft anchor constructs. Disclosed herein are various methods and soft anchor constructs to add multiple repair locations, while limiting the addition of sutures and volume/bulk.

### Definitions

Described herein are tissue repair systems, which use a soft anchor. The tissue repair systems of this disclosure provide a high fixation strength, fixing the soft anchor within bone. The tissue repair systems preferably knotlessly lock in a repaired configuration, avoiding the need for the surgeon to tie a knot. The tissue repair systems may include at least one suture. The term “suture” may include traditional sutures, that may be either hollow or may include braids along their core, unless were specified. The term “suture” may include equivalent flexible members, such as but not limited to suture tape or flattened suture and may in some cases be cable, ribbon or wire, where appropriate.

“Soft Anchor” is intended to mean a flexible and/or deformable anchor, where the anchor body is formed of soft, flexible suture-like material, that changes to a more laterally and/or radially expanded configuration upon setting or deployment. A tensioning member that is operatively coupled through a portion of the soft anchor may be tensioned to laterally expand the soft anchor. The term “soft anchor” does not preclude it from including supplemental portions that are rigid. In some embodiments, the soft anchor is formed entirely of braided strands. Some soft anchors deform to a deployed configuration that also includes a longitudinal contraction.

“Deploy” is intended to mean to change the shape of the body of the soft anchor so that it is set, fixed or anchored with/within a tissue. Deploying may increase a outer lateral dimension of the anchor body to secure it with a tissue. For example, this may fix the soft anchor within a bone hole. To deploy an anchor changes the anchor to a deployed configuration.

“Lock” or “locked configuration” with respect to a suture construct is intended to mean locking a suture such that the suture may no longer slide in at least one direction. Sliding in this at least one direction for example may loosen a repair tissue that is secured in place. The suture may form a loop including a tissue coupled thereto and the loop perimeter is prevented from sliding and increasing in perimeter size. With respect to an anchor body, a locked configuration is intended to mean locking the anchor body in a deployed configuration to inhibit the anchor body from relaxing/moving out of the deployed configuration.

“Knotlessly locking” or a word stemming derivative therefrom such as knotless locking or knotlessly locked, for example, is intended to mean a lock in a surgical construct or anchor system formed without having the tie a knot. A system provided with a pre-formed knot may be defined as knotlessly locking. A system configured to route the suture during operation of the surgical construct to form a knot, is also defined as knotlessly locking. Knotlessly locking may also be achieved by passing at least one suture along a tortuous route through small openings, or through a suture locking passage construct, may also be called Chinese finger traps, finger cinches or locking splices for example. To knotlessly lock the system, some of the sutures may extend through the suture locking passage of either the same suture or another suture, to form a self-locking adjustable suture construct as described herein. Suture locking passage may be selectively elongated, by applying tension to the locking passage to cinch around the suture disposed therein, thereby locking a portion of the adjustable suture construct.

“Transfer suture” is a flexible member that transfers another flexible member, such as a suture through the anchor construct. Transfer member may be a suture, suture tape or wire.

“Deploying suture” is intended to mean an elongate flexible member that may be a suture(s) (or equivalent as defined herein) that deploys the soft anchor body, usually upon tension being applied to the deploying member. In some embodiments, the deploying member may also provide other functions.

“Repair Suture” is the suture (or equivalent as defined herein) passed through the repair tissue and used to affix the repair tissue to bone.

### SUMMARY

Described herein are various improved methods and devices for tissue repair with a knotlessly locking construct, including a soft anchor. These and other features and advantages will be apparent from a reading of the following detailed description and a review of the associated drawings. It is to be understood that both the foregoing general description and the following detailed description are explanatory only and are not restrictive of aspects as claimed.

A first example knotless tissue repair construct is disclosed, the construct including an anchor body formed of a soft, suture-like material and has a proximal end, a distal end, and a longitudinal axis therealong. The construct also includes a repair suture with a first end, a second end and at least a length segment of the repair suture may be cannulated. The first end may be fixedly coupled to the anchor body, the length segment may be interwoven along and through a first sidewall of the anchor body and the repair second end may extend proximally from the anchor body proximal end. The construct also includes a transfer suture threaded through and along a second sidewall of the anchor body, into the length segment defining an entrance location, along and within the length segment that is interwoven along and through the first sidewall of the repair suture. The transfer suture also extends through or punches through the repair suture at a location adjacent the length segment entrance, defining a repair suture punch.

In some example embodiments, tension on at least one of the transfer suture or repair suture may change the anchor body to a deployed configuration. In the deployed configuration, the transfer suture may be operatively coupled to the repair suture second end, and tension on the transfer suture may draw the repair suture second end back through the deployed anchor body. Tension on the transfer suture may draw the repair suture second end though the repair suture punch adjacent the length segment entrance and then into and along the length segment and thereby forming a locking passage. Tension on the transfer suture may draw the repair suture second end and form a knotlessly locked repair loop. The repair suture may be fixedly coupled to the anchor body with two axially overlapping knots formed by the repair suture first end and the two axially overlapping knots may be external to the anchor body and adjacent the repair suture punch. The transfer suture may punch through the repair suture between the two axially overlapping knots and the entrance location. The anchor body may be tubular and include a lumen, and the transfer suture may be threaded through and along the second sidewall of the anchor body forming a plurality of weaving passes between an external anchor body surface, through braids of the second sidewall, while remaining spaced from the lumen. Weaving while avoiding the lumen may provide a high friction repair loop construct, that may increase resistance to loosening during cyclic loading. The repair suture may also interweave through the anchor body sidewall defining a plurality of passes, and at least one of the plurality of passes may extend from an external side surface of the anchor into the first sidewall and back out to the external side surface, without entering the lumen. The transfer suture may extend around an outer distal-most surface of the anchor body before punching through the repair suture, such that the anchor body distal-most surface provides a bearing a surface for the transfer suture, which may reduce stresses on the punch and a length segment entrance. This may mitigate damage to the punch and length segment entrance via tensions on the transfer suture during anchor deployment. The repair suture may interweave through the anchor body first sidewall for a first plurality of passes and the transfer suture may interweave through the anchor body second sidewall for a second plurality of passes, that are greater than the first plurality of passes.

Another example knotless tissue repair construct is disclosed, the construct including an anchor body formed of a soft material and having a proximal end, a distal end, and a longitudinal axis. The construct also includes a repair suture having a first end, a second end and a length segment that is cannulated. The first end is fixedly coupled to the anchor body, the length segment is interwoven along and through a first sidewall of the anchor body and the second end extends proximally from the anchor body proximal end. The transfer suture is threaded through and along a second sidewall of the anchor body, through and along the length segment of the repair suture and also punches through the repair suture adjacent a distal end of length segment, defining a repair suture punch. Tension on at least one of the transfer suture or repair suture changed the anchor body to a deployed configuration. The transfer suture may have a looped end that may operatively couple to the repair suture second end and draw the repair suture second end back through the anchor body, when it is in the deployed configuration. The transfer suture may draw the repair suture second end so that the repair suture punches through itself adjacent the length segment. The transfer suture may draw the repair suture second end through the length segment to form a locking passage, when it is in the deployed configuration, and thereby form a knotlessly locking repair loop.

In some embodiments the repair suture may be fixedly coupled to the anchor body with two axially overlapping knots formed by the repair suture first end, the two axially overlapping knots external to the anchor body and distal to the repair suture punch. The anchor body may be tubular and include an inner lumen, and the transfer suture may be threaded through and along the second sidewall of the anchor body forming a plurality of weave passes between an external anchor body surface, through braids of the second sidewall, while remaining spaced from the inner lumen. This weaving while avoiding the inner lumen may provide a high friction repair loop construct, resistant to loosening. The repair suture may interweave through the anchor body sidewall defining a plurality of passes, and at least one of the plurality of passes may extend from an external side surface of the anchor body into the first sidewall and back out to the external side surface, without entering the inner lumen. The transfer suture may extend around an outer distal-most surface of the anchor body before punching through the repair suture. The repair suture may interweave through the anchor body first sidewall for a first plurality of passes and the transfer suture may interweave through the anchor body second sidewall for a second plurality of passes, the first and second plurality of passes may be different in number.

Another example knotless tissue repair construct is disclosed, the construct including an anchor body formed of a soft material and having a proximal end, a distal end, and a longitudinal axis. The construct also includes a repair suture having a first end, a second end and a length segment that is cannulated. The first end is fixedly coupled to the anchor body, the length segment is interwoven along and through a first sidewall of the anchor body and the second end extends proximally from the anchor body proximal end. The construct also includes a transfer suture that is threaded along a path that includes through and along a second sidewall of the anchor body, around a distal-most surface of the anchor body, through an entire thickness of the repair suture, defining a repair suture punch and then through and along the length segment of the repair suture.

In some example embodiment, tension on at least one of the transfer suture or repair suture may change the anchor body to a deployed configuration. After deployment, the repair suture may be coupled to a repair tissue and then coupled to the transfer suture. Once coupled, tension on one end of the transfer suture may slide the transfer suture through the anchor body and also draw or shuttle the repair suture second end back through the anchor body, so that the repair suture punches through itself adjacent the length segment. Tension may also shuttle the repair suture second end into and along the length segment, thereby forming a locking passage and a knotlessly locking repair loop. The repair suture may be fixedly coupled to the anchor body with two axially overlapping knots formed by the repair suture first end, the two axially overlapping knots external to the anchor body and adjacent the repair suture punch. The transfer suture may punch through the repair suture between the two axially overlapping knots and the length segment. The anchor body may be tubular and include an inner lumen. The repair suture may interweave through the anchor body sidewall defining a plurality of passes, and wherein at least one of the plurality of passes may extend from an external side surface of the anchor body into the first sidewall and back out to the external side surface, without entering the inner lumen. The repair suture may interweave through the anchor body first sidewall for a first plurality of passes and the transfer suture may interweave through the anchor body second sidewall for a second plurality of passes, greater than the first plurality of passes.

Another example knotless all-suture anchoring system is disclosed, this system providing multiple fixation points. The system may include an all-suture or soft anchor body with a single repair suture interwoven therethrough. The single repair suture may be formed as a single continuously braided length of flexible material and has a first and second limb, both of which extend from a proximal end of the anchor body. The single repair suture has a lumen therealong. The system includes a first transfer suture repeatedly interwoven through the anchor body, including entering the single repair suture lumen for a length that is coextensive with the anchor body, so as to define a first locking passage length. The first transfer suture also includes two limbs extending directly from the proximal end of the anchor body. The system also includes a second transfer suture repeatedly interwoven through the anchor body, including entering the single repair suture lumen for a length, defining a second locking passage length, the second shuttling suture having two limbs extending directly from the proximal end of the all-suture anchor. At least one of the first and second transfer sutures are operatively coupled to a deployment mechanism of an insertion instrument, and tension on at least one of the first and second transfer sutures changes the anchor body to a deployed configuration within a target tissue. A first limb of the single repair suture limbs, once coupled to a first repair tissue may be drawn back through both the anchor body and first knotless locking length by the first transfer suture, while the anchor body is in the deployed configuration. A second limb of the single repair suture limbs is configured to couple to a second repair tissue before being drawn back through both the anchor body and second knotless locking length by the second transfer suture while the anchor body is in the deployed configuration.

In some embodiments the first and second knotless locking length are the same segment of the repair suture. The first limb of the single repair suture and the second limb of the single repair suture may both drawn through the anchor body serially. The first knotlessly locking length may be located towards a proximal end of the anchor body and the second knotlessly locking length may be disposed distally from the first knotlessly locking length along the repair suture.

An example method of tissue repair with a knotless tissue repair construct is also disclosed herein. The method may include obtaining any of the knotless tissue repair constructs disclosed herein and inserting it into a target tissue. The anchor body may then be deployed by applying tension to a transfer suture, so as to set the anchor body with the target tissue. The repair suture first limb may be coupled to a repair tissue after the anchor body has been deployed. The repair suture first limb may be coupled to the first transfer suture. The first transfer suture may then be drawn through the anchor body while in a deployed configuration and thereby withdraw the repair suture first limb through the anchor body and through the first knotlessly locking length to form a first locking passage. The repair suture first limb may then be tensioned to knotlessly locking the construct.

In some example methods the suture second limb may be coupled to another repair tissue after the anchor body has been deployed and the repair suture second limb may then be coupled to the second transfer suture. The second transfer suture may then be withdrawn through the anchor body while in a deployed configuration to withdraw the repair suture second limb through the anchor body and through the second knotlessly locking length to define a locking passage. The repair suture second limb may then be tensioned to knotlessly locking the construct. Tensioning the repair suture first and second limb may both be done simultaneously, after both repair suture limbs have been withdrawn and both locking passages have been formed. The repair tissue and the another repair tissue may be different locations along the same tissue. The repair tissue and the another repair tissue may be different tissues.

### BRIEF DESCRIPTION OF THE DRAWINGS

The disclosure will be more fully understood by reference to the detailed description, in conjunction with the following figures, wherein:

FIG. 1A schematically shows an example prior art knotlessly locking soft anchor construct; disclosure;

FIG. 1B schematically shows a close up of a fixed end of the example prior art knotlessly locking soft anchor construct shown in FIG. 1A;

FIG. 2A schematically shows a first example knotlessly locking soft anchor construct, in accordance with this disclosure;

FIG. 2B schematically shows a close up of an example fixed end of the first example knotlessly locking soft anchor construct, in accordance with this disclosure;

FIG. 2C schematically shows the first example knotlessly locking soft anchor construct, in a deployed and knotlessly locked configuration, in accordance with this disclosure;

FIG. 2D schematically shows another close up of another example fixed end of the first example knotlessly locking soft anchor construct, in accordance with this disclosure;

FIGS. 3A-3C schematically show knotlessly locking soft anchor constructs with other example fixation means or constructs, in accordance with this disclosure;

FIGS. 4A-4B schematically show knotlessly locking soft anchor constructs a splice knot, in accordance with this disclosure;

FIG. 4C schematically shows a splice knot in a tightened configuration, in accordance with this disclosure;

FIG. 5 schematically shows an example of a low profile knotlessly locking soft anchor construct that couples to multiple repair locations, in accordance with this disclosure;

FIG. 6 schematically shows another example of a low profile knotlessly locking soft anchor construct that couples to multiple repair locations, in accordance with this disclosure;

FIG. 7 schematically shows another example of a low profile knotlessly locking soft anchor construct that couples to multiple repair locations, in accordance with this disclosure;

FIG. 8 schematically shows another example of a low profile knotlessly locking soft anchor construct that couples to multiple repair locations, in accordance with this disclosure;

FIG. 9 schematically shows another example of a low profile knotlessly locking soft anchor construct that couples to multiple repair locations, in accordance with this disclosure;

FIG. 10 schematically shows the low profile knotlessly locking soft anchor construct shown in FIG. 9 in a deployed and knotlessly locked configuration, in accordance with this disclosure;

FIG. 11 schematically shows another example of a low profile knotlessly locking soft anchor construct that couples to multiple (three) repair locations, in accordance with this disclosure;

FIG. 12A schematically shows an example three-loop suture construct, in accordance with this disclosure; and

FIG. 12B schematically shows an example anchor with the three-loop suture construct assembled thereto, in accordance with this disclosure.

### DETAILED DESCRIPTION

In the description that follows, like components have been given the same reference numerals, regardless of whether they are shown in different examples. To illustrate example(s) in a clear and concise manner, the drawings may not necessarily be to scale and certain features may be shown in somewhat schematic form. Features that are described and/or illustrated with respect to one example may be used in the same way or in a similar way in one or more other examples and/or in combination with or instead of the features of the other examples.

As used in the specification and claims, for the purposes of describing and defining the invention, the terms “about” and “substantially” are used to represent the inherent degree of uncertainty that may be attributed to any quantitative comparison, value, measurement, or other representation. The terms “about” and “substantially” are also used herein to represent the degree by which a quantitative representation may vary from a stated reference without resulting in a change in the basic function of the subject matter at issue. “Comprise,” “include,” and/or plural forms of each are open ended and include the listed parts and can include additional parts that are not listed. “And/or” is open-ended and includes one or more of the listed parts and combinations of the listed parts. Use of the terms “upper,” “lower,” “upwards,” and the like is intended only to help in the clear description of the present disclosure and are not intended to limit the structure, positioning and/or operation of the disclosure in any manner.

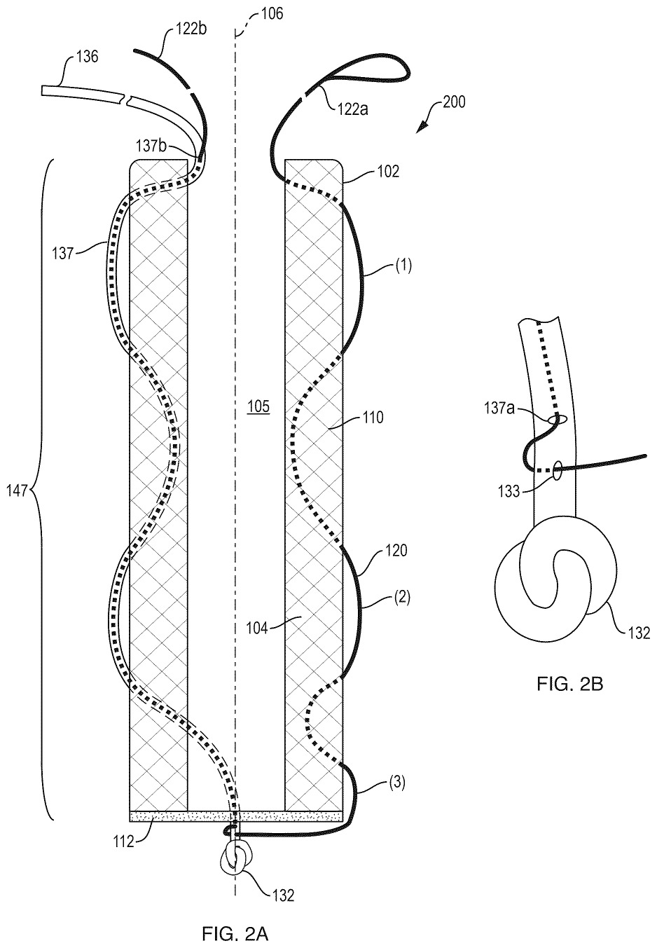

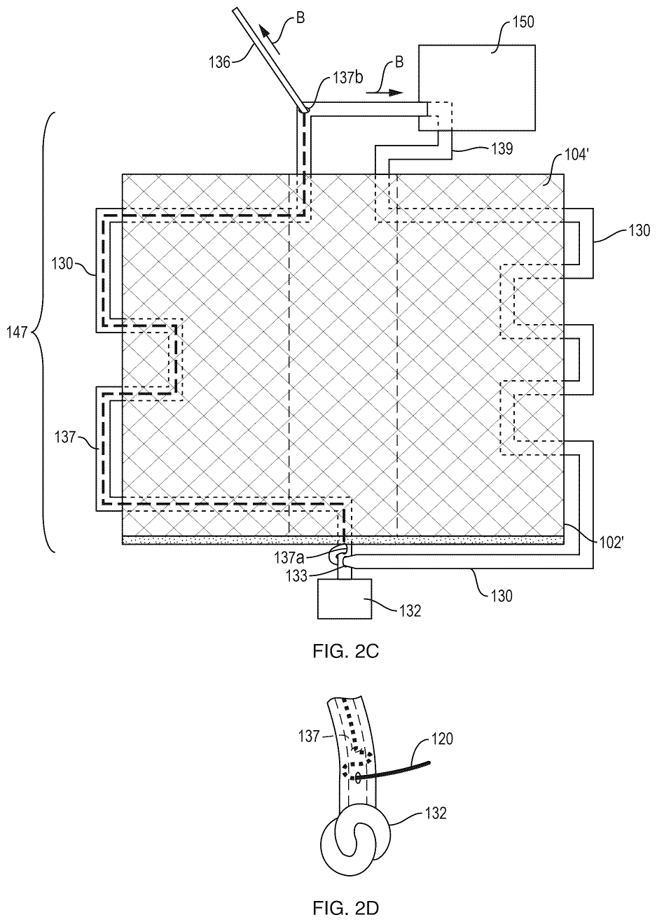

A first example knotlessly locking soft anchor construct 200 that may provide strong fixation and maintains this strong fixation with use and time is illustrated in FIG. 2A. Construct 200 is similar to construct 100 except where noted and like components are given the same numerical indicator. Soft anchor 102 may be like anchors disclosed in at least U.S. Pat. No. 9,962,149, titled “Tissue Repair Assembly”; commonly owned and herein incorporated by reference in its entirety. Soft anchor 102 may be obtained operatively coupled with an insertion system that actively deploys anchor 102, producing a deployed anchor with a strong fixation strength within or with tissue when deployed. Active deployment as disclosed in U.S. Pat. No. 9,962,149, titled “Tissue Repair Assembly” provides a back stop to hold the anchor 102 in place while deploying the anchor 102 via a high tensioning load on the deploying sutures, to produce high fixation within the target tissue when the anchor is deployed. Soft anchor 102 may define a tubular braided body 104, that may include a closed or partially closed distal end 112. Tubular body 104 may be formed of braided strands 110. Distal end 112 may be at least partially sealed, using adhesive or heat sealing.

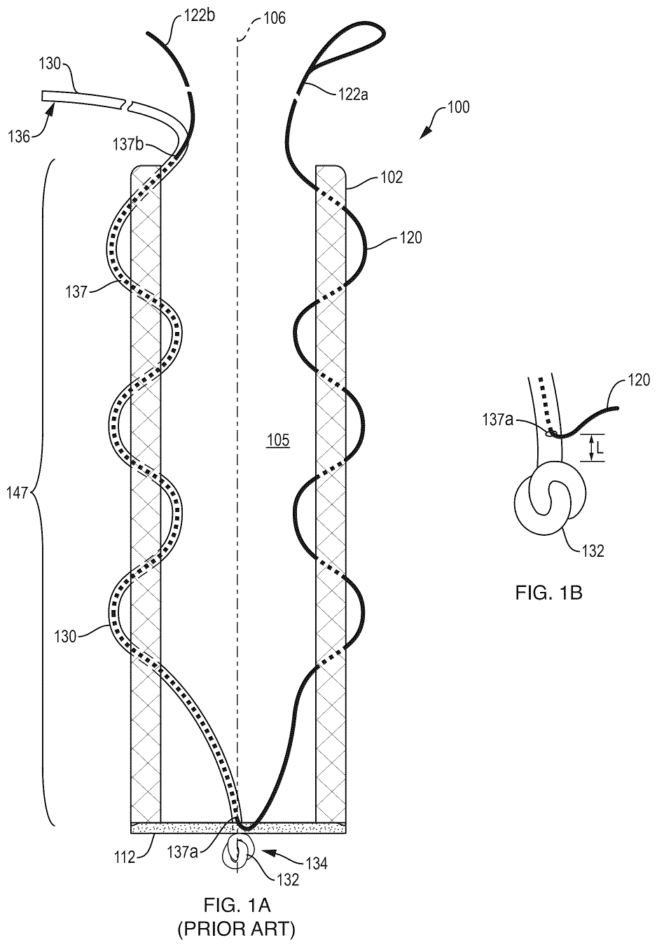

Similar to the embodiment shown in FIG. 1A, construct 200 may include a transfer suture 120 that interweaves repeatedly through walls of the anchor body 104. Transfer suture 120 may extend along a first side of the longitudinal axis 106 of the body 104, around and external to the distal end 112 and return along a wall on an opposing side of the axis 106. Having the transfer suture 120 extend both around and external to the distal end 112 may shield the punch (133, disclosed later herein) and the locking passage entrance 137a from damage induced by the application of high deployment loads via the transfer suture 120 to the construct 200. As explained herein, the transfer suture 120 may deploy the anchor via tension on the transfer suture ends (122a, 112b), the higher this tension, the stronger the anchor fixation and less likely this fixation will reduce over time and use. However, these high-tension loads may tear into the cannulation entrance 137a. Shielding may help preserve the integrity of the repair suture fixation means. Stated another way, when the transfer suture 120 extends around and external to the distal end 112, the distal end edge of the body 104 provides a bearing surface for the transfer suture 120. Without this bearing surface, the high tension loads along the transfer suture 120 during anchor deployment may tear the cannulation entrance 137a and punch 133, and lead to a damaged construct and reduced locking passage length. Therefore, the inventors have found that this additional bearing surface helps to mitigate damage to the construct 200, while still permitting high tension loads; high tension loads providing a strong anchor fixation within the target tissue.

Two limbs 122a, 122b of transfer suture 120 may extend from a proximal end 116 of anchor 102. A first limb 122a may terminate with a snare loop. Transfer suture 120 may be slidingly coupled to anchor body 104. Of note, in this embodiment, transfer suture 120 may extend only partially through walls of the tubular body 104, so as to interweave through the wall, turning short of reaching the inner wall of lumen 105 for at least one of the passes or weaves through the tubular wall. In this figure, two passes are shown, that are short of reaching the lumen 105. Having the transfer suture 120 remain within the wall of the anchor body 104 and preferably absent from the lumen 105 has been found to increase friction in the constructs final, locked and deployed configuration. While too much friction may lead to higher than desired loads to shuttle the repair suture to form the repair loop (described hereinafter), some friction is beneficial in that it provides a supplemental resistance to loosening of the locking passage 147 during post-op cyclic loading. After surgery, the repair may be cyclically loaded as the person moves, which may loosen a knotlessly locked configuration. The additional friction incurred by weaving the transfer suture 120 (and thereby the repair suture after shuttling) mostly within the body sidewall, may help to counteract loosening.

Repair suture 130, separately formed from the transfer suture 120 may be fixedly coupled to anchor body 104 via knot 132. As discussed herein, knot 132 may prevent repair suture 130 from being removed from anchor body 104. Repair suture 130 may extend along lumen 105 of tubular body 104 at the proximal and distal ends of body 104 but may interweave between the proximal and distal ends (116, 112 respectively), again preferably without extending along the lumen 105 for at least one of the passes or weaves through the anchor body tubular wall.

As obtained, shown in FIG. 2A, transfer suture 120 may make three passes (1, 2, 3) through the anchor body 104 as it interweaves along the transfer suture side of the anchor body 104, while the locking passage side of the anchor body (left hand side on the figure) may make less than three passes. Repair suture 130 may make only 2 passes though the tubular wall on the locking passage side of the anchor body. The more passes, the higher the friction along the final repair loop construct. However, too much friction may induce a higher load to shuttle the repair suture 130 through the anchor body 104 and cannulation 137. Therefore, the locking passage 147 may pass through the anchor sidewall a reduced number of time than the transfer suture side, to reduce this resistance. In this example, the locking passage 147 passes twice through the anchor body 104, to balance the shuttling loads through the locking passage 147 with the preferred higher friction of the final repair loop. The number of passes depends on the suture sizes and anchor configurations. Therefore, the number of locking passage passes are configured to provide sufficient friction to the knotlessly locked configuration, while providing an acceptable, functional shuttling load on the user.

In this embodiment, looking at close-up figure FIG. 2B, the transfer suture 120 extends around an external surface of the distal end 112 and then may form a punch 133. This punch 133 may extend all the way through the repair suture thickness from a first external side to a second opposing external side, at a location immediately proximal of the knot 132. In some embodiments, the transfer suture 120 may punch through the knot 132 (not shown). Transfer suture 120 may then pass partially through the repair suture to enter cannulation 137, defining entrance 137a, similar to embodiment construct 100. Therefore, this embodiment includes an extra pass or punch 133 of the transfer suture 120 through the repair suture 130, that may be between knot 132 and locking passage entrance 137a.

This routing may improve knotless locking by increasing the friction in the construct at the distal end of the locking passage 147. This increase in friction may help mitigate loosening of locking passage 147 that may allow slippage within the locking passage 147, particularly when the knotless locking loop is used to capture a significant amount of soft tissue. In addition, the punch 133 may shield knot 132 from the higher fixation loads on the repair construct. The punch 133 may bolster the fixation means of the repair suture fixed end and prevent or inhibit the knot 132 from further cinching. In some alternate embodiments, the punch may be a partial punch and may not extend all the way through the entire thickness of the repair suture 130, an example of which is shown in FIG. 2D. In this example shown in FIG. 2D, the transfer suture 120 may form a tortuous path along and between strands of the repair suture 130, along sidewalls of the repair suture, and may remain within the repair suture 130, entering directly into the cannulation 137 without first exiting the repair suture 130.

In addition to or instead of punch 133, repair suture end 134 may be ultrasonically welded to anchor body 104. In addition to or instead of punch 133, knot 132 may be pre-tensioned with a tool to a high load to cinch the knot 132 and reduce likelihood of slippage. Knot 132 may be further secured by application of heat to fuse the suture fibers together and resist slippage. A tail end stopper could be formed using a heat knife for example, where the end immediately beyond the knot 132 could be melted into an amorphous globular mass, leading to the knot resisting slippage. This effectively creates a stopper for the stopper knot 132.

FIG. 2C illustrates construct 200 in a deployed, repaired and knotless locked configuration, hereinafter final configuration. Deployment may not only laterally increase the anchor body 104 but may also longitudinally reduce the anchor length (from distal end to proximal end) and may also increase the wall thickness of the tubular body 104, such that the space defined by lumen 105 is filled, or substantially reduced with the braided walls. In some embodiments, the space defined by the lumen is completely filled with the braided walls. In order to reach this final configuration, the anchor 102 may be first deployed via tension on at least one of the transfer suture 120 or repair suture 130. The repair suture 130 may then be operatively coupled to tissue 150 before being threaded through snare loop at limb end 122a (see FIG. 2A). Drawing on end 122b of transfer suture 120 may then slide the transfer suture 120 through the deployed anchor body 104′, concomitantly removing transfer suture 120 from the deployed anchor body 104′ and replacing it with the repair suture 130. Transfer suture 120 therefore may be defined as both a deploying suture and a transfer suture. Withdrawing suture end 122b may transfer the repair suture 130 into and along the deployed anchor body 104′, so that the repair suture 130 traces the same path defined by the transfer suture 120 when first obtained. The resulting final configuration is a repair suture 130 that is fixedly coupled at a distal end 112, coupled to a repair tissue 150 and woven around and through the deployed anchor body 104′, as schematically shown in FIG. 2C. Tension (arrows B) on the repair suture 130 either from suture end 136 or from resulting loop portion 139 may act to shrink the diameter of cannulation 137 and cinch the repair suture 130 around itself, in a similar fashion to a Chinese finger trap. This may further lock the construct 200.

FIG. 2C illustrates a simplified more circuit like illustration of the system 200 for simplicity of understanding. The tortuous path that the repair suture 130 takes both through the deployed anchor body 104′ and through itself along cannulation 137 provides sufficient friction to lock the repair suture 130 in place and prevent it from sliding or loosening. The deployed anchor body 104′ is also knotlessly locked in the deployed configuration. The repair suture 130 in the final configuration may now punch through itself at punch 133 that may be adjacent knot 132, before entering cannulation entrance 137a. This punch 133 may bolster/shield knot 132 as explained herein and improve the tension on the locking passage 147. The repair suture 130 may pass through a first side of anchor body 104′ making 3 passes, interweaving through the anchor body wall, but turning short of reaching the lumen 105, for at least one of the passes through the anchor sidewall. This may increase the friction along the knotless loop construct as described herein. The repair suture 130 may wrap around a distal-most end, externally to anchor 120 to shield the cannulated entrance 137a degradation due to the high deploying and fixation loads. The repair suture 130 may pass through the other side of anchor body 104′ making 2 interweaving passes, while also within the cannulation 137, interweaving through the anchor wall, but turning short of reaching the lumen 105 for at least one of the passes through the tubular wall. This may increase the friction along the knotless loop construct and in particular along the locking passage 147, as described herein.

Deploying the anchor body 104 may move the locking passage 147 from being entirely within the anchor body 104 to protruding proximally therefrom, such that exit 137b may be proximally spaced from anchor body 104. Some of the suture ends (either suture 120 and/or 130) may be tapered in diameter at their ends, which may advantageously reduce the load required to draw the repair suture 130 into and along the body 104 and cannulation 137.

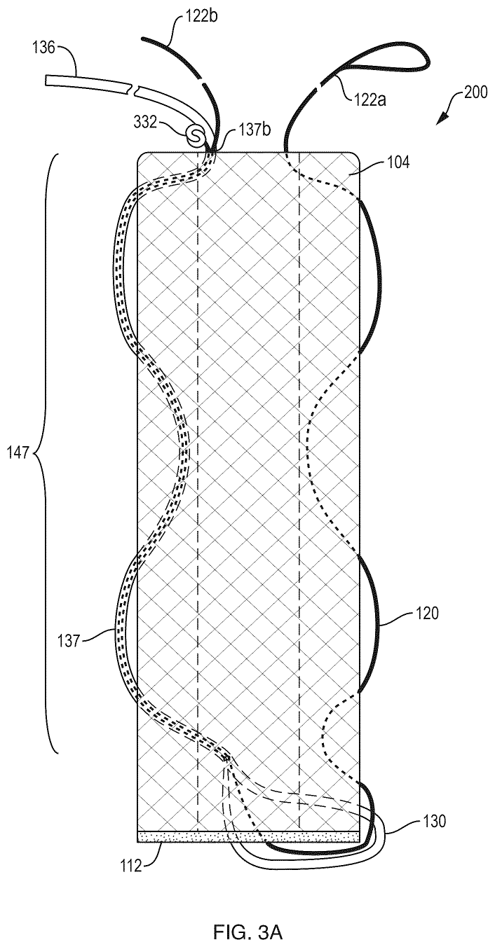

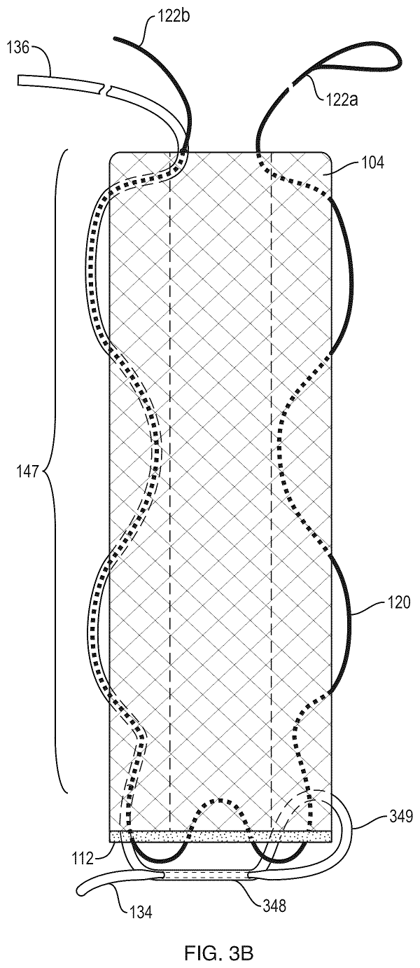

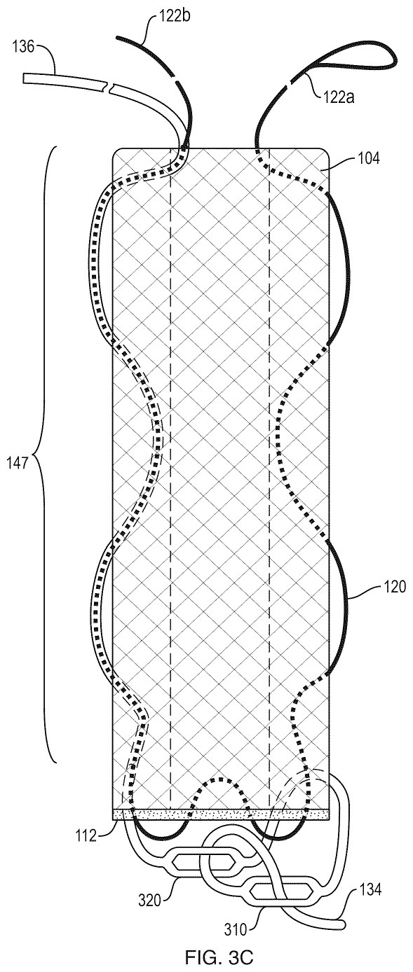

FIG. 3A-3C illustrate schematic representations of other embodiments of knotlessly locking constructs that may provide more robust attachments of the repair suture 130 to the anchor body 104. All three are simplified for ease of understanding and may be like construct 100 except when noted and like components are given the same numerical indicator. All three figures show the construct as obtained, before the repair suture 130 is shuttled through the anchor body 104. FIG. 3A illustrates an alternative routing of repair suture 130, wherein the repair suture 130 is looped around the distal end of the anchor body 104 and then doubled back into the cannulation 137. This embodiment may utilize the locking nature of the cannulation (locking passage 147) to secure the repair suture 130 to the anchor body 104. Repair suture 130 may include a knot 332 at a proximal end of locking passage 147. Repair suture 130 may loop around and interweave between braids of the anchor braided wall at the distal end 112.

FIG. 3B illustrates a repair suture 130 interwoven through one side of the anchor body 104 and then back through a distinct, second locking passage 348. This second locking passage 348 is configured to fix the repair suture 130 to the anchor body 104. Second locking passage 348 may be free of a transfer suture 120. Second locking passage 348 may form a loop 349, as shown, that extends from a distal end of the locking passage 348. In other embodiments, loop 349 may loop around and enter locking passage 348 from the opposite end. Second locking passage 348 is free of a transfer suture 120. Repair suture 130 may form a loop 349 that interweaves through the anchor braided wall distal end 112. Tension on end 134 may then fix repair suture end with anchor distal end 112. The advantage of the second locking passage 348 is that the end of the splice does not move with tightening, like a hitch knot. The end is defined by the entrance (or exit) of the suture only. In FIG. 3C, the repair suture 130 may be punched through itself twice (310, 320) creating a tortuous path to prevent the repair suture 130 from retreating. These punches are shown in a loose loop, to show the suture path, but may be two serial punches similar to punch 133. The repair suture 130 may pass between the braided strands of the anchor distal end 112 before the two punches 310, 320.

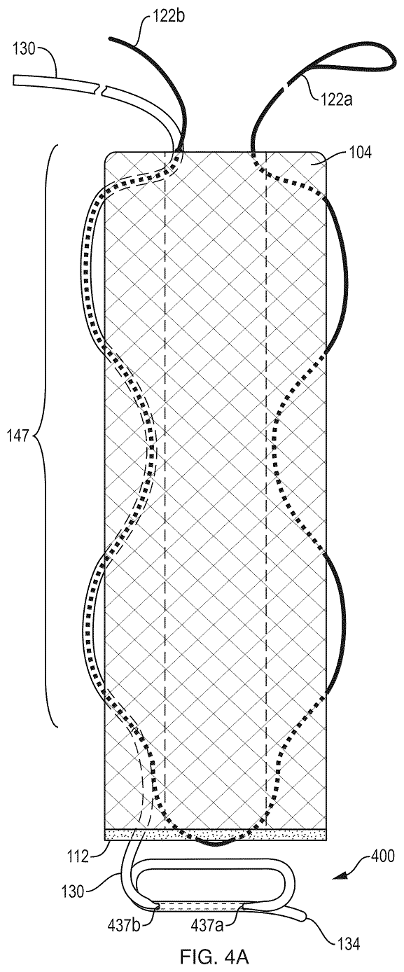

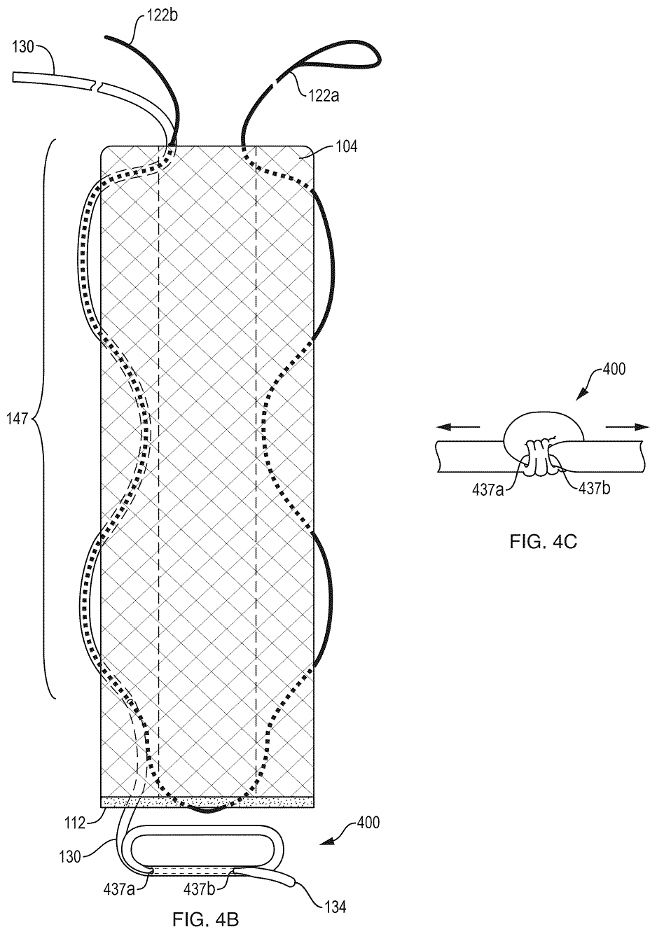

FIGS. 4A-4B illustrate a other splice knot embodiments 400 that may be an alternative to knot 132. Splice knot 400 includes a cannulated length 437, with an entrance 437a and exit 437b, similar to locking passage 147. Splice knot 400 may provide a knot with a fixed location along the suture 130, that cannot slip like a regular knot. The location is defined by either the entrance or exit (437a, 437b). FIG. 4A illustrates a first example splice knot routing for repair suture 130, where repair suture 130 entrance 437a is distal of exit 437b. FIG. 4B illustrates the reverse wherein the repair suture 130 entrance 437a is proximal exit 437b. FIGS. 4A and 4B illustrate knot configurations with an open or loose loop configuration, to show routing. However, in practice, end 134 and/or repair suture 130 is tensioned to form a final knot 400, as shown in FIG. 4C. Final knot shown in FIG. 4C is a tightened version of routing shown in FIG. 4B. Opposite to a finger trap where the locking passage portion is tensioned to reduce its diameter and cinch around the suture disposed within the passage, in this splice knot 400, the passage portion is shortened or concertina-ed, forming a large volume knot with a defined end, via the entrance or exit from cannulated length 437. The cannulated length becomes shorter.

### Knotless Anchor Construct for Repairing Multiple Locations.

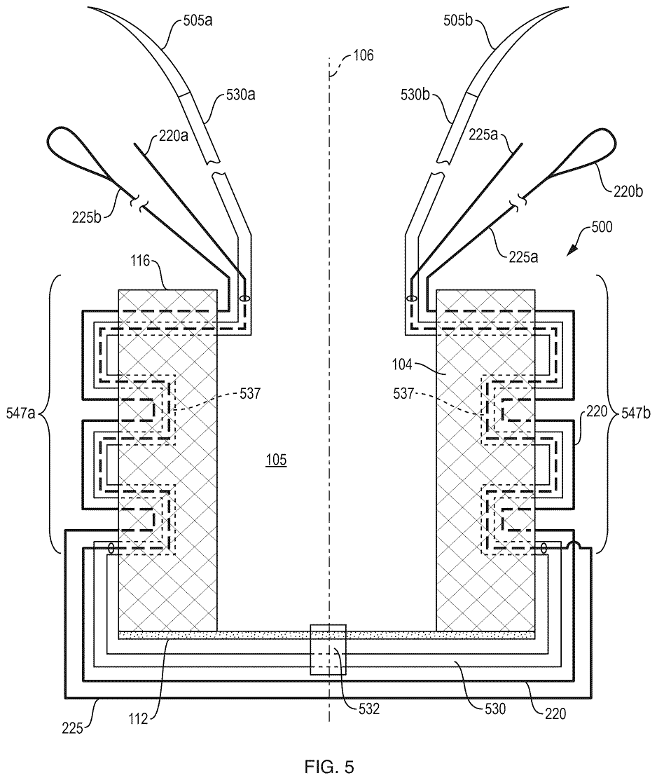

FIG. 5 illustrates a low-profile knotless anchor construct 500, in that it is a construct that provides two repair suture ends 530a, 530b from a single repair suture 530. Each end 530a, 530b provides a fixation or repair location that may separately form a repair loop in different fixation locations, that may be spaced away from each other. The two fixation locations may be to the same tissue at different locations, or two separate tissues. This system may independently couple to multiple fixation locations and may also independently knotlessly lock. While the inventors contemplated duplicating the two sutures 120, 130 disclosed in previous embodiments disclosed herein, they found that adding the two extra sutures (extra transfer and repair suture) along the soft anchor 102 added significant volume and complexity, and therefore was less preferable. Construct 500 provides a lower volume (low profile) solution to a multiple repair location knotless anchor construct.

Remaining on FIG. 5, like components to construct 100 are given the same identifier. Construct 500 may include anchor body 104, a single repair suture 530 (which may be similar to repair suture 130), a first transfer suture 220 and a second transfer suture 225. Repair suture 530 may be cannulated, for forming locking passage(s) at selective locations therealong, similar to repair suture 130. Construct 500 is shown in a configuration as obtained or located, before deploying the anchor body 104 and shuttling the repair suture ends 530a, 530b. The single repair suture 530 may interweave through both sides of the anchor body 104 such that both ends 530a, 530b extend from the anchor proximal end 116. In some embodiments each end 530a, 530b may be preassembled to a needle 505a, 505b respectively. First transfer suture 220 may interweave through anchor body 104 and also into and along a portion of the cannulation 537 of repair suture 530, defining a first knotless locking length 547a of the system. Second transfer suture 225 may interweave through anchor body 104 and also along the cannulation 537 or lumen of the same repair suture 530, defining a second knotless locking length 547b. First and second knotless locking length 547a, 547b may be on opposing sides of the anchor body 104. Construct 500 therefore includes two repair suture ends 530a, 530b, wherein each end (530a, 530b) may be individually coupled to a repair tissue and separately drawn through the anchor body 104 to knotlessly lock with the single anchor body 104. Body 104 is preferably set within a target bone before coupling and shuttling the repair suture ends 530a, 530b to the target tissue(s). Repair suture 530 may be fixedly coupled to anchor body 104, approximately midway along the repair suture 530. Repair suture 530 may include a knot 532, the knot may include a loop that loops between the anchor body braided strands, to maintain the knot in a central location along the anchor body axis 106. The two locking passages 547a, 547b may each interweave through the anchor body 104, on opposing sides of anchor lumen 105 to each other. The repair suture 530 may interweave partially through the walls, avoiding the lumen 105 for at least one pass. Repair suture ends 530a and 530a may be shuttled by either transfer suture 220 or 225.

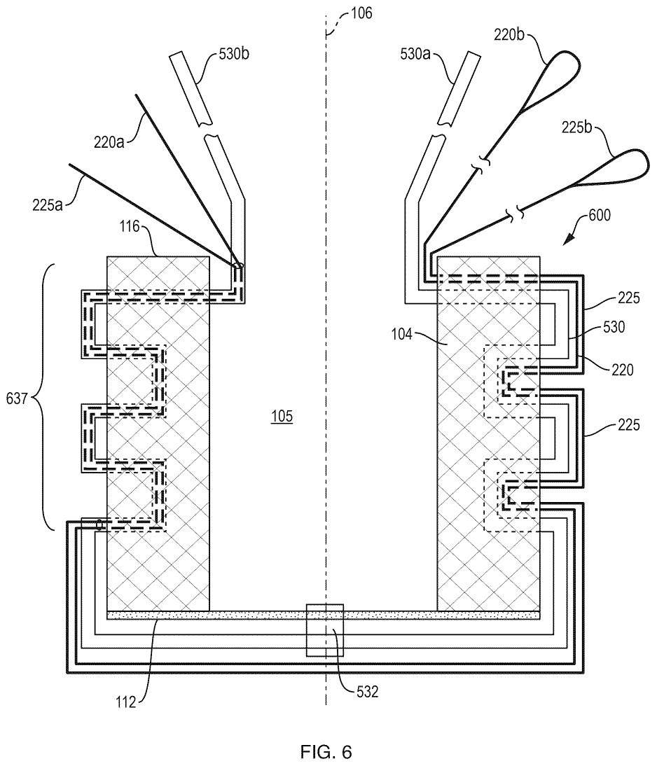

Illustrated in FIG. 6 is another example low-profile knotless anchor construct 600, similar to at least construct 500 and like components to construct 100 and 500 are given the same identifier. Construct 600 has a single locking passage 637, and both repair suture ends 530a, 530b are shuttled through this single locking passage 637. Each repair suture end 530a, 530b may be sequentially coupled to a tissue or tissues and then sequentially shuttled through the repair suture cannulation 537, that is the same location along the repair suture 530. Locking passage 637 therefore may cinch around two length of repair suture 530. Repair suture ends 530a, 530b are first shuttled from a proximal end to a distal end, through the anchor body 104 and then upon turning and being shuttled from the distal end 112 to the proximal end 116, enter the cannulation 537. This preferably disposes the locking passage 637 at the end of the formed knotless locking loop, such that the locking passage is directly adjacent the end of the locking loop as the repair suture end (530a, 530b) exits the anchor body 104.

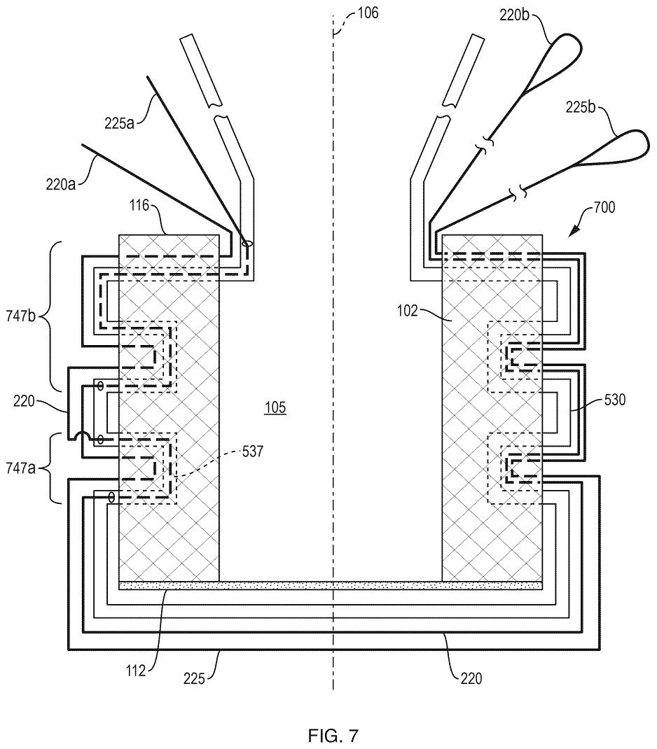

Illustrated in FIG. 7 is another example low-profile knotless anchor construct 700, similar to at least construct 100, 500 or 600 and like components to these constructs are given the same identifier. Construct 700 has a two locking passages 747a, 747b. Each repair suture end 530a, 530b may be sequentially or simultaneously coupled to a tissue or tissues and then simultaneously or sequentially shuttled or drawn through the repair suture cannulation 537. The two locking passages 747a, 747b may be serially arranged along the repair suture 530, along the same side axis 106 of anchor body 104.

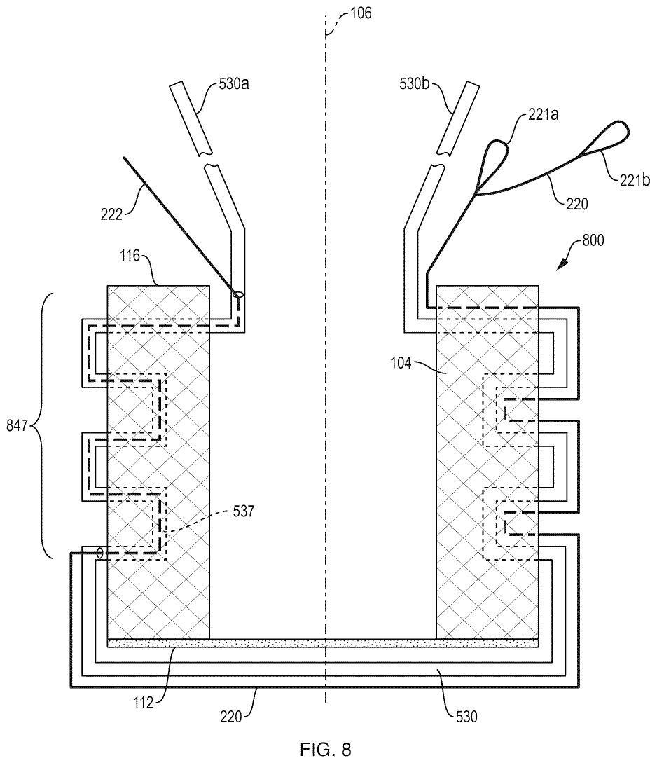

Illustrated in FIG. 8 is another example low-profile knotless anchor construct 800, similar to previous constructs disclosed herein and like components to these constructs are given the same identifier. Construct 800 has a single knotless locking passage 847 and a single transfer suture 220. The single transfer suture 220 with two loop ends 221a, 221b, that may be offset from each other along the transfer suture 220. During the repair, after the anchor 104 has been deployed, a first repair suture end 530a may be coupled to a tissue at a first location and then coupled to first loop end 221a of transfer suture 220. The second repair suture end 530b may be coupled to the tissue at a second, separate location, or to a different tissue before being coupled to second loop end 221b and then both repair suture ends 230a, 230b may be drawn into the knotless construct. Having the two repair sutures drawn with a single transfer suture may further reduce volume of the construct as obtained. Having the two loops (221a, 221b) slightly offset from each other may reduce the step increase in volume of suture as it is drawn between the strands of the deployed anchor 104′ and the cannulation 537. This may help to stagger the volume increase being introduced into the construct as a whole and more specifically into the repair suture lumen at the entrance to the locking passage 837. This may reduce the force required to draw both repair suture ends 530a, 530b into the deployed anchor body 104′ and repair suture cannulation 537.

If the system is provided with needles, the transfer suture 220 (or sutures 220, 225) may be operatively coupled to an anchor deployment mechanism, while the repair suture 530 may be housed in a separate location, separated from the deployment mechanism. An example insertion instrument that may separately house repair sutures may be similar to system disclosed in International Patent application number PCT/US21/34590, titled “Tissue Repair System”, filed May 27, 2021, this patent application commonly owned and herein incorporated by reference in its entirety.

A method of use may therefore include inserting the knotless construct (500, 600, 700) into a target tissue with an insertion instrument and deploying the anchor body 104. Deploying may include applying tension, that may be applied via an insertion instrument, to transfer suture 220 (and/or 225 where present). Once the anchor body 104 is deployed within the target tissue, the repair suture ends 530a, 530b (and pre-attached needles 505 where present) may be removed from the insertion instrument. The first repair suture end 530a may be coupled to a first repair tissue. After the first repair suture end 530a has been coupled to the first repair tissue, needle 505 may be removed, and the repair suture end 530a coupled to a first looped end of the transfer suture 220. Drawing on a second end of the transfer suture 220 may then slide the first repair suture end 530a through deployed anchor body 104′, while removing the transfer suture 220 therefrom. This may place the first repair suture end 530a along a tortuous path through the deployed anchor body 104′, as disclosed herein that may form a knotlessly locking configuration.

The second repair suture 530b may be coupled to another location of the first repair tissue, or another tissue. After the second repair suture end 530b has been coupled, the corresponding needle 505 (if present) may be removed, and the second repair suture end 530b coupled to a first looped end of the other transfer suture 225. Drawing on a second end of the transfer suture 225 may then slide the second repair suture end 530b through deployed anchor body 104′, while removing the other transfer suture 225 from the construct. This may place the second repair suture end 530b along a tortuous path through the deployed anchor body 104′, as disclosed herein that may form a knotlessly locking configuration. In some example methods the transfer sutures 220, 225 may be coupled to their respectively repair suture ends (530a, 530b) and withdrawn simultaneously.

In some example embodiments repair suture 530 may not be provided preassembled with needles. In some example embodiment, repair suture 530 may include a taper at each end. For example, repair suture 530 may taper down from a larger suture size to a 2-0 section at both free ends, for the last 2 to 10 inches. The middle length portion of the repair suture 530 may have a cross section similar to a No. 2 suture. This tapered down section may allow for easier passing of the repair suture 530 through itself at the entrance to the locking passages (547, 747 for example).

Another method of use may include inserting the knotless construct 800 into a target tissue with an insertion instrument and deploying the anchor body 104. Deploying may include applying tension, that may be applied via an insertion instrument, to transfer suture 220. Once the anchor body 104 is deployed within the target tissue, the repair suture ends 530a, 530b (and pre-attached needles 505 if present) may be removed from the insertion instrument. The first repair suture end 530a may be coupled to a first repair tissue. After the first repair suture end 530a has been coupled to the first repair tissue, needle 505 may be removed (if present), and the repair suture end 530a coupled to a first looped end 221a of the transfer suture 220. The second repair suture end 530b may be coupled to another location along the first repair tissue, or another tissue. After the second repair suture end 530b has been coupled, needle 505 may be removed (if present), and the repair suture end 530b coupled to a second looped end 221b of the transfer suture 220. Drawing on a second end 222 of the transfer suture 220 may then slide both the first repair suture end 530a and second repair suture end 530b through deployed anchor body 104′. Drawing on a second end 222 of the transfer suture 220 may draw the first repair suture end 530a and second repair suture end 530b serially through deployed anchor body 104′ slightly offset from each other, defined by the offset between looped ends 221a, 221b. This may now place both repair suture ends 530a, 530b through the same locking passage 847 and the construct 800 is in a knotless locking configuration. Further tension on repair suture ends 530a, 530b may knotless lock the construct 800 similar to other embodiments disclosed herein.

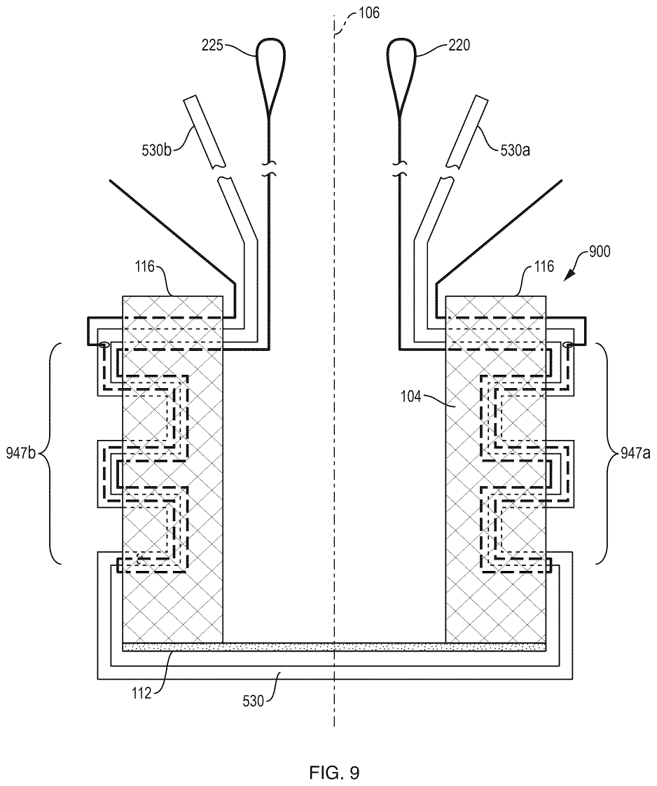

FIG. 9 schematically shows another example knotless construct embodiment 900, similar to at least construct 500. In the embodiment repair suture 530 may define a cannulation 537, along which a portion of a first and second transfer suture 220, 225 extend, defining a first locking passage 947a and second locking passage 947b. Similar to previously disclosed embodiments, after coupling the repair suture ends 530a, 530b to tissue or tissues, they may be coupled to the looped ends of transfer suture 220, 225 similar to methods disclosed herein. Ends 530a, 530b may then be drawn into the deployed anchor body 104′ and cannulation 537 of repair suture 530. If no needles are preassembled, all the suture ends transfer 220, 225 and repair ends 530a, 530b may be operatively coupled to a deployment mechanism of an inserter. Tension on all of these suture ends may deploy the anchor body 104 into a tighter bunched up shape, that may improve fixation of the anchor body 104 within the target tissue. In this embodiment, locking passages 947a, 947b may extend only part way along the anchor body 104, leaving a proximal most portion of anchor body 104 free of any locking passage. Locking passages 947a, 947b may be sufficiently short to limit any proximal protrusion of the locking passages 947a, 947b from the anchor proximal end, after anchor deployment (and anchor shortening). Since the anchor body 104 is generally placed immediately below the cortical bone layer or is tensioned to engage the underside of this cortical bone layer, any protrusion of the locking passages 947a, 947b may be unsightly. In addition, any protruding locking passage portion may extend out of the bone hole, causing possible concern for local tissue irritation, and palpability through the skin.

Also shown in FIG. 9, and different to construct 500, the each transfer suture is limited to its own side. Stated another way, the first transfer suture 220 is limited to a first side of anchor body 104 only, and a second transfer suture 225 is limited to a second side only of anchor body 104. This reduces any overlapping of pathways of the two repair loop constructs over each other, that may help avoid any snagging or entangling between the first and second repair loop constructs; entangling or snagging possibly hindering shuttling and or knotless locking. More specifically transfer suture 220 is assembled to the construct 900 so that when it draws the repair suture 530 through the construct 900 along the following path; first it may draw the repair suture end 530a along and through the anchor body 104, limited to a first side of the anchor longitudinal axis 106 to the anchor distal end 112. This path may extend adjacent to the repair suture locking passage 947a, located on the same side of anchor body axis 106. Then it may draw the repair suture end 530a into the locking passage 947a at a location adjacent anchor distal end 112, and then draw it proximally along the repair suture cannulation 537 so that the repair suture end 530a exits the locking passage 947a at a location spaced distally from a proximal most end 116 of anchor. The path continues so that the repair suture end 530a may interweave and pass through the anchor body 104 in a proximal direction before exiting anchor proximal end 116. Transfer suture 220 shuttles repair suture end 530a along a single side of anchor body 104. Similarly, transfer suture 225 is assembled to the construct 900 so that when it draws the repair suture second end 530b through the construct 900, it may mirror the path taken by end 530a limited to the opposing side of the anchor longitudinal axis 106. End 530b may be drawn first along the anchor body 104 to the anchor distal end 112, then drawn into the second locking passage 947b at a location adjacent the anchor distal end 112, the be drawn proximally along the repair suture cannulation 537 and exit the second locking passage 937b at a location spaced distally from a proximal most end 116 of anchor. The second end 530b may be then drawn so as to interweave through and along the anchor body 104 in a proximal direction before exiting anchor proximal end 116. Transfer suture 225 shuttles second repair suture end 530b along a single side of anchor body 104, that may be diametrically opposite to (opposing side of longitudinal axis 106) the pathway defined by transfer suture 220.

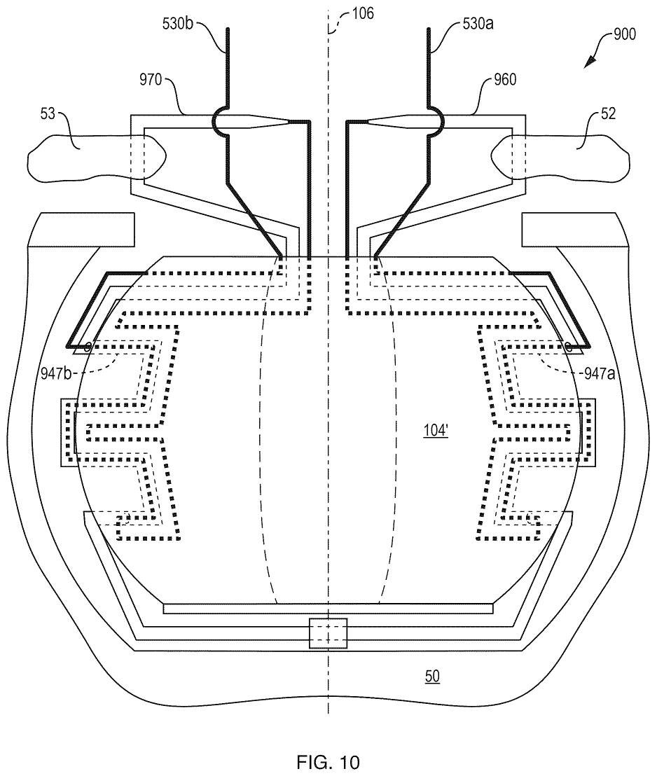

FIG. 10 illustrates construct 900 in a final configuration. Anchor body 104 is deployed within a target bone 50. As shown, bone is spaced slightly away from anchor body 104, to keep the figure clear and show the suture routing. However, in reality, lateral edges of the anchor and the interweaving repair suture portions are embedded into the cancellous bone, deforming the softer cancellous bone, while the harder cortical (top) layer remains relatively unchanged. In final configuration, a first repair end 530a is coupled to a first repair tissue 52 and also drawn through the deployed anchor body 104′ and through itself. FIG. 10 also illustrates construct 900 with a second repair end 530b coupled to a second tissue 53 (that may be a different portion of the first tissue 52 or a separate tissue) and also drawn through the deployed anchor body 104′ and through itself along a second opposing side. This forms a first repair loop 960 and a second repair loop 970. Repair suture 530 is shown transitioning to a narrower diameter at a location between the repair tissue (52, 53) and the anchor body 104′. Repair suture 530 may taper and have smaller diameter ends to reduce the loads required to shuttle the repair suture ends 530a, 530b though the anchor body 104 and cannulation 537. In other embodiments the taper may transition at closer to each end (530a, 530b). The first loop 960 may be formed by first coupling, for example, a first repair suture end 530a of repair suture 530 to a target repair tissue 52, followed by coupling the repair suture end 530a to looped end of first transfer suture 220 and then drawing the first end 530a into the deployed anchor body 104″, and then into an end of locking passage 947a using transfer suture 220. The first repair loop 960, once completed, routes the end 530a around or through the repair tissue 52, back into and along the deployed anchor body 104′ on a first side of the deployed anchor body 104′, into the locking passage 947a at an anchor distal end 112, and along passage 947a so that the end 530a extends from a proximal end of locking passage 947a and deployed anchor body 104′. Tension on end 530a may cinch the locking passage 937a and knotlessly lock the first repair loop 960. In some embodiments the first repair loop 960 once completed, routes the first end 530a around the tissue 52, back into and along the deployed anchor body 104′ on a first side of the anchor, interweaving along the sidewall on a first side of anchor longitudinal axis 106 up until a distal end 112 of deployed anchor body 104′ and then into a distal end of locking passage 947a, exiting at a proximal end of locking passage 937a before interweaving along the remains of deployed anchor body 104′ towards the anchor proximal end 116. In some embodiments, construct 900 may include a punch, similar to punch 133 at the distal end of each locking passage (947a, 947b). A second repair loop 970 may be formed in a similar fashion on the opposing side of deployed anchor body 104′.

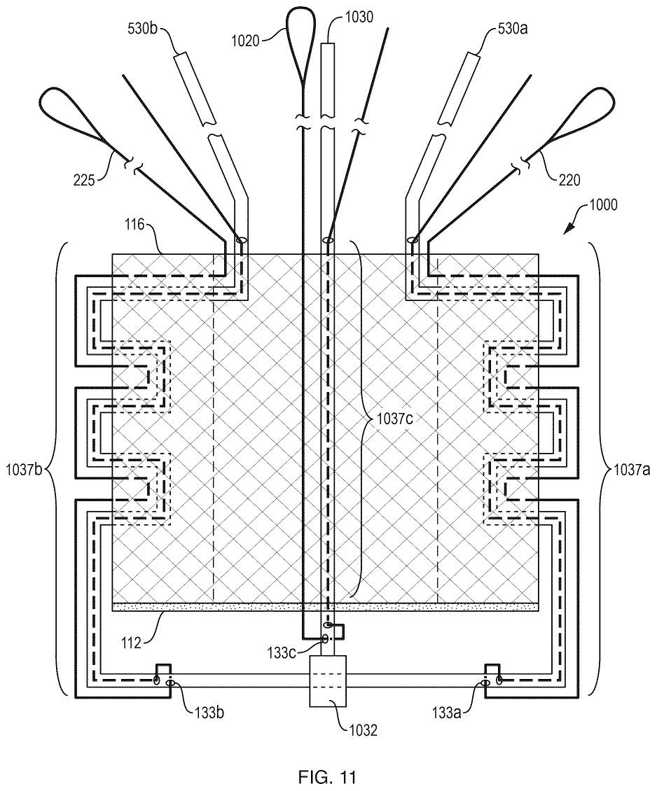

FIG. 11 illustrates another knotless locking construct with a single anchor body 104 and three repair suture ends, 530a, 530b and 1030. At least two of the repair suture ends may be ends of a single suture, such as suture ends 530a, 530b disclosed herein. A third repair suture 1030 may be fixedly coupled to either the anchor body 104 and/or the other repair suture(s). In other embodiments, three separate repair sutures are assembled to anchor body 104. Repair sutures may be fixedly coupled to each other, and/or to anchor body 104 so as to limit movement of each locking passage (1037a, 1037b and 1037c) relative to the anchor body 104. Repair sutures (530, 1030) may be fixedly coupled to each other, and/or to anchor body 104 via fixed coupling means as disclosed herein, such as via a knot, splice knot, welding, adhesive for example. Construct may include three corresponding transfer sutures 220, 225 and 1020 that may be preassembled to an insertion instrument deployment mechanism to deploy anchor body 104. At least one of the transfer sutures 220, 225 and 1020 is preferably coupled to the deployment mechanism. Transfer sutures 220, 225 and 1020 may also each form a separate repair loop construct, similar to previous embodiments disclosed herein. Each transfer suture may selectively couple to a corresponding repair suture end to shuttle the repair suture through the deployed anchor body 104′ and through a repair suture locking passage (1037a, 1037b. 1037c). In addition, similar to embodiments previously disclosed, at least one of the transfer sutures (all three shown) may first punch through a corresponding repair suture thickness adjacent a point of entry into the repair suture locking passage (1037a, 1037b, 1037c) similar to punch 133 disclosed in at least construct embodiment 200. In this embodiment each transfer suture 220, 225 and 1020 punches through a corresponding repair suture portion (133a, 133b, 133c), directly adjacent a corresponding entrance into a repair suture cannulation.

Third repair suture end 1030 may extend solely along anchor lumen 105. This maintains a separation of the third repair suture from the other repair constructs and may avoid snagging. This may also help to keep the construct volume smaller. Embodiment 1000 may form 3 separate knotlessly locking repair loops that may be coupled to 3 different tissues, or three portions of the same tissue for example.

### Multi-Loop Transfer Suture

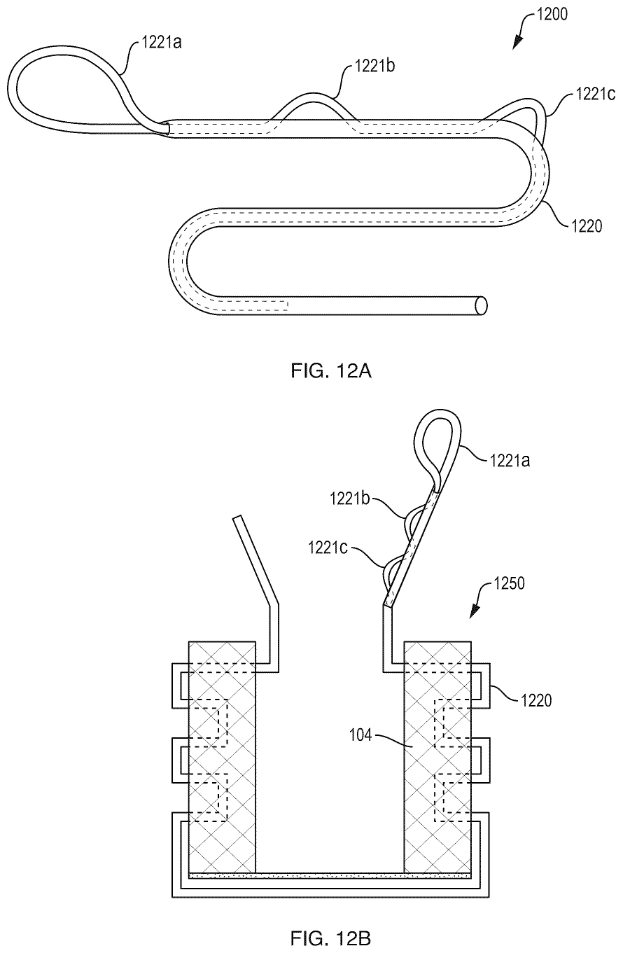

Construct 800 disclosed a transfer suture 220 with multiple loops 221a, 221b. Transfer suture loops are preferably formed in a smooth manner, so as to avoid introduction of bulges or discontinuities, that may increase the force to shuttle other sutures through a construct. Forming loops with knots for example may form these bulges. Multiple loops 221a, 221b may be formed during the manufacturing process, and the strands may be bifurcated for a length, before recombining to form a single length. Examples of bifurcated braids may be disclosed in at least US Application number 20220233302, titled Methods And Devices For Tissue Graft Fixation, filed June 2020. Another means of forming a suture with multiple loops is illustrated in FIG. 12a, which may include an example suture construct 1220 with a plurality of axially spaced spliced loops 1221a, 1221b and 1221c. In this figure, 3 spliced loops are shown, but two spliced loops for embodiments 800 for example, or more spliced loops for other constructs may be formed. Construct 1220 may be a transfer suture, configured to shuttle, in this example construct, at least 3 suture ends. In some embodiments, each splice may also include a punch, similar to punch 133, to prevent each splice loop 1221a, 1221b and 1221c from slipping and changing size.

A transfer suture with multiple loops, such as construct 1220 may draw repair suture(s) though any anchor body. A transfer suture such as 1220 may draw repair suture(s) though cannulations of a suture, to form locking passages, as disclosed herein. FIG. 12B illustrates transfer suture such as 1220 interwoven through anchor body 104. Transfer suture such as 1220 with multiple loops may be woven along different paths and through different soft anchor configuration. In some embodiments, this construct 1250, as shown may be provided absent a repair suture. The repair suture(s) may then be selected by the clinician and may include larger sutures and/or suture tape. The clinician may then draw these selected sutures through the anchor body 104, to couple the repair sutures thereto. The clinician may then draw these selected sutures through the anchor body 104 after the soft anchor has been deployed. The clinician may, in this example therefore couple three separate and selectable sutures to the anchor body 104 after deployment. Coupling the repair sutures in an axially spaced arrangement along the transfer suture construct 1220 may reduce the force required to shuttle the repair sutures through the deployed anchor body 104, since each suture may be sequentially introduced into the deployed anchor body 104′, which may gradually dilate the spaces between braids of the anchor for example. Furthermore, in embodiments that include knotless locking constructs such as some of the constructs disclosed herein, sequentially drawing suture ends may reduce the force required to shuttle the repair sutures into and through the suture cannulations.

The inventors have found that shutting larger sutures into the anchor body 104 after deployment may allow the insertion instrument and initial volume of the anchor as provide to be small, therefore requiring a smaller drill opening or tissue opening to insert it. Stated in the alternative, should three large sutures be provided preassembled into the anchor and along the insertion device, the anchor and insertion tubes may be larger in diameter initially to accommodate the increased number of components, that may require larger tissue openings.

One skilled in the art will realize the disclosure may be embodied in other specific forms without departing from the spirit or essential characteristics thereof. The foregoing examples are therefore to be considered in all respects illustrative rather than limiting of the disclosure described herein. Scope of the disclosure is thus indicated by the appended claims, rather than by the foregoing description, and all changes that come within the meaning and range of equivalency of the claims are therefore intended to be embraced therein.

What is claimed is:

1. A knotless tissue repair construct comprising:
an anchor body formed of a soft material, the anchor body having a proximal end, a distal end, and a longitudinal axis;
a repair suture having a first end, a second end and a cannulated length, the first end fixedly coupled to the anchor body, the cannulated length interwoven along and through a first sidewall of the anchor body and the second end extending proximally from the anchor body proximal end; and
a transfer suture threaded through and along a second sidewall of the anchor body, into and along the cannulated length of the repair suture and also punches through the repair suture at the anchor body distal end, to form a tortuous path before entering a distal end of the cannulated length.

2. The knotless tissue repair construct of claim 1 wherein tension on at least one of the transfer suture or repair suture is configured to change the anchor body to a deployed configuration and when in the deployed configuration the transfer suture is configured to operatively couple to the repair suture second end and draw the repair suture second end back through the anchor body, through the cannulated length and also punches through itself along the tortuous path adjacent the cannulated length, thereby forming a knotlessly locked repair loop.

3. The knotless tissue repair construct of claim 1 wherein the repair suture is fixedly coupled to the anchor body with a knot formed by the repair suture first end, the knot external to the anchor body, the transfer suture punching though the repair suture between the knot and anchor body distal end, external to the anchor body.

4. The knotless tissue repair construct of claim 3 wherein the transfer suture punches through the repair suture between the knot and the cannulated length distal end.

5. The knotless tissue repair construct of claim 1 wherein the anchor body defines an inner lumen, and wherein the transfer suture is threaded through and along the second sidewall of the anchor forming a plurality of weave passes between an external anchor body surface, through the second sidewall, while remaining spaced away from the inner lumen, so as to increase friction along the locked repair loop.

6. The knotless tissue repair construct of claim 1 wherein the anchor body defines an inner lumen, and wherein the repair suture interweaves through the anchor body sidewall defining a plurality of passes, and wherein at least one of the plurality of passes extends from an external side surface of the anchor body into the first sidewall and back out to the external side surface, without entering the inner lumen.

7. The knotless tissue repair construct of claim 1 wherein the transfer suture extends around an outer distal-most surface of the anchor body before punching through the repair suture, such that the distal-most surface provides a bearing surface to reduce stresses on an entrance into the cannulated length.

8. The knotless tissue repair construct of claim 7 wherein the repair suture interweaves through the anchor body first sidewall for a first plurality of passes and wherein the transfer suture interweaves through the anchor body second sidewall for a second plurality of passes, the second plurality of passes more than the first plurality of passes.

9. A knotless tissue repair construct comprising:
an anchor body formed of a soft material, the anchor body having a proximal end, a distal end, and a longitudinal axis;
a repair suture having a first end, a second end and a cannulated length, the first end fixedly coupled to the anchor body, the cannulated length interwoven along and through a first sidewall of the anchor body and the second end extending proximally from the anchor body proximal end; and
a transfer suture threaded through and along a second sidewall of the anchor body, through and along the cannulated length of the repair suture and also punched through the repair suture at the anchor body distal end, distal to the cannulated length, defining a repair suture punch;
wherein tension on at least one of the transfer suture or repair suture is configured to change the anchor body to a deployed configuration and wherein in the deployed configuration, the transfer suture is configured to operatively couple to the repair suture second end and draw the repair suture second end back through the anchor body, so that the repair suture punches through itself adjacent the cannulated length, thereby forming a knotlessly locked repair loop.

10. The knotless tissue repair construct of claim 9 wherein the repair suture is fixedly coupled to the anchor body with two axially overlapping knots formed by the repair suture first end, the two axially overlapping knots external to the anchor body and distal to the repair suture punch.

11. The knotless tissue repair construct of claim 9 wherein the anchor body defines an inner lumen, and wherein the transfer suture is threaded through and along the second sidewall of the anchor forming a plurality of weave passes between an external anchor body surface, through braids of the second sidewall, while remaining spaced from the inner lumen, configured to provide a high friction repair loop construct.

12. The knotless tissue repair construct of claim 9 wherein the anchor body defines an inner lumen, and wherein the repair suture interweaves through the anchor body sidewall defining a plurality of passes, and wherein at least one of the plurality of passes extends from an external side surface of the anchor into the first sidewall and back out to the external side surface, without entering the inner lumen.

13. The knotless tissue repair construct of claim 9 wherein the transfer suture extends around an outer distal-most surface of the anchor body before punching through the repair suture.

14. The knotless tissue repair construct of claim 13 wherein the repair suture interweaves through the anchor body first sidewall for a first plurality of passes and wherein the transfer suture interweaves through the anchor body second sidewall for a second plurality of passes, more than the first plurality of passes.

15. A knotless tissue repair construct comprising:
an anchor body formed of a soft material, the anchor body having a proximal end, a distal end, and a longitudinal axis;
a repair suture having a first end, a second end and a cannulated length, the first end fixedly coupled to the anchor body, the cannulated length interwoven along and through a first sidewall of the anchor body and the second end extending proximally from the anchor body proximal end; and
a transfer suture threaded through and along a second sidewall of the anchor body, around a distal-most surface of the anchor body, through an entire thickness of the repair suture, defining a repair suture punch located at the anchor body distal end and then through and along the cannulated length of the repair suture.

16. The knotless tissue repair construct of claim 15 wherein tension on at least one of the transfer suture or repair suture is configured to change the anchor body to a deployed configuration and wherein the transfer suture is configured to operatively couple to the repair suture second end and draw the repair suture second end back through the anchor body, wherein in the deployed configuration, so that the repair suture punches through itself adjacent the cannulated length, thereby forming a knotlessly locked repair loop.

17. The knotless tissue repair construct of claim 15 wherein the repair suture is fixedly coupled to the anchor body with two axially overlapping knots formed by the repair suture first end, the two axially overlapping knots external to the anchor body and adjacent the repair suture punch.

18. The knotless tissue repair construct of claim 17 wherein the transfer suture punches through the repair suture between the two axially overlapping knots and the cannulated length.

19. The knotless tissue repair construct of claim 15 wherein the anchor body defines an inner lumen, and wherein the repair suture interweaves through the anchor body sidewall defining a plurality of passes, and wherein at least one of the plurality of passes extends from an external side surface of the anchor into the first sidewall and back out to the external side surface, without entering the inner lumen.

20. The knotless tissue repair construct of claim 15 wherein the repair suture interweaves through the anchor body first sidewall for a first plurality of passes and wherein the transfer suture interweaves through the anchor body second sidewall for a second plurality of passes, more than the first plurality of passes.
